(19) United States
(12) Patent Application Publication
(10) Pub. No.: US 2014/0276059 A1
(43) Pub. Date: Sep. 18, 2014

(54) EXTERNALLY IMAGING A BODY STRUCTURE WITHIN A PATIENT

(71) Applicant: VOLCANO CORPORATION, San Diego, CA (US)

(72) Inventor: David Sheehan, Poway, CA (US)

(73) Assignee: VOLCANO CORPORATION, San Diego, CA (US)

(21) Appl. No.: 14/203,662

(22) Filed: Mar. 11, 2014

Related U.S. Application Data
(60) Provisional application No. 61/777,155, filed on Mar. 12, 2013.

(51) Int. Cl. A61B 8/12; A61B 8/08

(52) U.S. Cl. CPC A61B 8/12; A61B 8/0891
USPC 600/443

(57) ABSTRACT

The invention generally relates to methods of imaging a body structure using an ultrasound device that is external to a patient's body. In certain aspects, the methods of the invention involve providing an ultrasound device that is external to a patient's body, and externally imaging a body structure within the patient using the external ultrasound device.

### RELATED APPLICATION

The present application claims the benefit of and priority to U.S. provisional patent application Ser. No. 61/777,155, filed Mar. 12, 2013, the content of which is incorporated by reference herein in its entirety.

### FIELD OF THE INVENTION

The invention generally relates to methods of externally imaging a body structure within a patient.

### BACKGROUND

Cardiovascular disease frequently arises from accumulation of atheromatous material on inner walls of vascular lumens, particularly arterial lumens of the coronary and other vasculature, resulting in a condition known as atherosclerosis. Atherosclerosis occurs naturally as a result of aging, but it may also be aggravated by factors such as diet, hypertension, heredity, and vascular injury. Atheromatous and other vascular deposits restrict blood flow and can cause ischemia which, in acute cases, can result in myocardial infarction. Atheromatous deposits can have widely varying properties, with some deposits being relatively soft and others being fibrous and/or calcified. In the latter case, the deposits are frequently referred to as plaque.

Conventional cardiovascular imaging includes the use of external imaging methods, such as x-ray angiography to image a vessel from the outside. Angiography is a medical imaging technique used to visualize a lumen of blood vessels and organs of the body, with particular interest in the arteries, veins and the heart chambers. This is traditionally done by injecting a radio-opaque contrast agent into the blood vessel and imaging using X-ray based techniques such as fluoroscopy.

Unfortunately, angiography presents risks to both the patient and the provider. Since the images are taken in real time, substantially greater amounts of x-ray radiation are required as compared to a radiograph (x-ray picture). In addition to the x-ray exposure, patients may suffer side effects from the radiopaque contrast agents, including pain, adverse drug interactions, and renal failure. For technicians and physicians, there are also risks of x-ray exposure as well as orthopedic injuries (e.g., lower back strain) due to the extra weight of the lead-lined aprons and other protective equipment.

### SUMMARY

The invention uses external ultrasound to image a body structure inside a patient's body. Ultrasound presents less risks to a patient and to an operator because it is conducted without the use of x-rays or radio-opaque dyes, while still providing the necessary resolution to construct an image of body structure being examined.

Methods of the invention are accomplished by providing an ultrasound device that is external to a patient's body, and externally imaging a body structure within the patient using the external ultrasound device. The collected data is then used to construct an image of the body structure that can be displayed on a display device. Methods of the invention may be used to image any body structure, and are particularly useful for imaging vessels of the cardiovascular system. In certain embodiments, the ultrasound emits a signal at about 20 megahertz or greater.

Methods of the invention may be combined with internally obtained characteristics of the body structure to produce a co-registered image. The image is constructed by combining external imaging data and internal data to produce the image of the body structure. The internal characteristics may be obtained by imaging inside the body structure, using any known intravascular imaging technique, such as intravascular ultrasound or optical coherence tomography. Alternatively or in combination, the internal characteristics may be obtained by fractional flow reserve.

### BRIEF DESCRIPTION OF THE DRAWINGS

FIG. 1 is a graphical illustration of a three dimensional length of artery, including a highly diseased segment.

FIG. 2 is a graphical illustration of a portion of the artery depicted in FIG. 1 with a longitudinal section removed along lines 2 to illustratively depict different elements of atherosclerotic plaque.

FIG. 3 is a graphical illustration of the artery from FIGS. 1 and 2 wherein an imaging catheter has been inserted in the artery.

FIG. 4 is detailed view of a section of the artery depicted in FIG. 3 including an imaging catheter in the artery.

FIG. 5 is a flowchart depicting a set of exemplary steps for creating a co-registered three-dimensional graphical display.

FIG. 6 illustratively depicts a vessel reconstruction graphical image based on image creation techniques embodied in a system and method incorporating the present invention.

FIG. 7 illustratively depicts a first graphic display in relation with a graphical representation of a three-dimensional or two-dimensional image.

FIG. 8 illustratively depicts a second graphic display in relation with a graphical representation of a three-dimensional or two dimensional image.

FIG. 9 illustratively depicts a third graphic display in relation with a graphical representation of a three-dimensional or two-dimensional image.

FIG. 10 illustratively depicts a graph including two separate sequences of values corresponding to lumen area, in relation to image frame number and linear displacement along an imaged vessel, prior to axial registration adjustment.

FIG. 11 illustratively depicts a graph including two separate sequences of values corresponding to lumen area, in relation to image frame number and linear displacement along an imaged vessel, after axial registration adjustment.

FIG. 12 illustratively depicts a graphical display of external ultrasound and vessel (e.g., IVUS) images on a single graphical display prior to axial registration adjustment.

FIG. 13 illustratively depicts a graphical display of external ultrasound and vessel (e.g., IVUS) images superimposed on a single graphical display after axial registration adjustment.

FIG. 14 illustratively depicts the process of circumferential registration of external ultrasound and vessel (e.g., IVUS) image sets.

FIG. 15 illustratively depicts angular image displacement in relation to circumferential registration of external ultrasound and vessel (e.g., IVUS) image sets.

FIG. 16 illustratively depicts a graph of actual and best fit rotational angle corrections displayed in relation to image frame number.

FIG. 17 show two external ultrasound images of vessels. B-mode (left) (cephalad to left) and M-mode (right) images of distal common carotid artery.

FIG. 18 is a set of ultrasound images showing ischemia-induced brachial artery (BA) reactivity test in healthy individual. Arm cuff was inflated at 200 mm Hg for 3 minutes. BA diameter responses (C to H) and pulsed wave (PW) Doppler velocity responses at baseline (A) and immediately on cuff release (B). Baseline 2-dimensional (2D) image of BA (C) with 30-(D), 60-(E), 90-(F), 120-(G), and 180-(H) second posthyperemia 2D images. Note increase in BA diameter in posthyperemia phase, compared with baseline, peaking at 90 milliseconds (F), and increase in flow and decrease in resistance immediately on cuff release (B) compared with baseline (A). Automated measurements of peak systolic velocity (white arrow), end-diastolic velocity (white arrowhead), peak velocity-time integral (green tracing), and mean velocity-time integral (blue tracing) are shown. PW Doppler assessment is made at 60-degree angle correction. BA diameter measurements are made at onset of QRS complex (C to H) (angled white arrows). Note fascial plane landmarks that are reproduced in each image (double-sided black arrows).

FIG. 19 is block diagram of a system of the invention for receiving and processing internal imaging data and external imaging data to produce a co-registered image.

FIG. 20 is a block diagram of a networked system for receiving and processing internal imaging data and external imaging data to produce a co-registered image.

### DETAILED DESCRIPTION

The invention generally relates to methods of externally imaging a body structure within a patient. In certain embodiments, methods of the invention involve providing an ultrasound device that is external to a patient's body, and externally imaging a body structure within the patient using the external ultrasound device. Methods of the invention may be used for carotid artery imaging and brachial artery reactivity testing (BART).

Any commercially available ultrasound system may be used with methods of the invention, such as those sold by GE healthcare, Philips Healthcare, Siemens, etc. Typically, the transducer used will be solid state transducer. A solid state (or phased array) transducer has no rotating parts, but instead includes an array of transducer elements (for example 64 elements). The transducer is generally a piezoelectric crystal that is intermittently excited with an electrical pulse. The excitation pulse causes the transducer to vibrate, sending out a series of transmit pulses. The transmit pulses are sent at a frequency that allows time for receipt of echo signals. The sequence of transmit pulses interspersed with receipt signals provides the ultrasound data required to reconstruct a complete cross-sectional image of a vessel. Further explanation of ultrasound is provided, for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et al., U.S. Pat. No. 4,917,097, Eberle et al., U.S. Pat. No. 5,135,486, and other references well known in the art relating to ultrasound devices and modalities.

Typically a clear water-based gel is applied to the area of the body being studied to help the transducer make secure contact with the body and eliminate air pockets between the transducer and the skin that can block the sound waves from passing into your body. The transducer is pressed firmly against the skin in various locations, sweeping over the area of interest or angling the sound beam from a farther location to see an area of concern better.

Methods of then invention may be used to evaluate the presence of obstructive atherosclerosis in the setting of symptomatic cerebrovascular disease or asymptomatic carotid bruit. Methods of the invention may also be used to measure intimalmedial thickness (IMT) and detect nonobstructive plaque to evaluate the relation of these findings to cardiovascular disease risk factors and cardiovascular disease morbidity and mortality. In addition, changes in carotid IMT may be used as a measure of efficacy of pharmacologic intervention.

Methods of the invention may be performed using standard ultrasound machines equipped with high-frequency transducers (e.g., usually 5-12 MHz and linear array but maybe greater, e.g. 20 MHz and above) and appropriate software. Standard transducers used in adult echocardiography (2.0-3.5 MHz) do not provide adequate near-field resolution for superficial vascular imaging. Ideally, the system should allow full-screen display of M-mode images if this modality is used in measurement of IMT and lumen diameters. Patient preparation and positioning are known in the art. In brief, the patient should be supine with slight hyperextension and rotation of the neck in the direction opposite the probe. The common carotid artery (CCA) is identified in the transverse or longitudinal plane and scanned from its origin to bifurcation. The internal carotid artery (ICA) and external carotid artery are identified using standard anatomic features. Scanning in the transverse plane and from multiple angles optimizes detection of nonobstructive plaque.

The IMT can be measured in the CCA, the bifurcation (bulb), and either of the branch vessels (usually the ICA). Because of its tubular shape, perpendicular location relative to the transducer beam, and virtually universal accessibility, measurement yield and reproducibility of the CCA IMT are higher than for the ICA or bulb IMT.

The IMT may be measured from the near (closest to the transducer) and/or the far wall. Excess gain or blossoming of the highly echogenic near-wall adventitia into the echolucent media, or of the echogenic near-wall intima into the echolucent lumen, will result in systematic under-measurement or over-measurement, respectively, if IMT of the near wall is measured. In contrast, incursion of echoes from the far-wall intima into the media will not influence overall IMT measured from the far wall. Thus measurement of the far wall is likely to be more accurate than measurement of the near wall.

In certain embodiments, IMT is measured from B-mode images (FIG. 19). Alternatively, B-mode-guided M-mode images of the distal CCA may be obtained (FIG. 19). In either case, because of the very small dimensions, wall thickness should be measured using computer assistance with electronic calipers or semiautomated edge-detection algorithms. Although spatial resolution is comparable with the two techniques, temporal resolution is far superior with M-mode imaging, thereby facilitating standardization of measurements at the time of minimum diameter, when the diastolic distending pressure is known, and estimation of pulsatility or vascular function. Instantaneous changes in pressure and diameter can be assessed from continuous tracing of M-mode images and simultaneous pressure waveforms of the contralateral carotid artery obtained using applanation tonometry.

Regardless of whether IMT is measured from B-mode or M-mode images, cyclic variations in IMT and lumen diameter should be taken into account by electrocardiographic gating and/or determination of minimal (end-diastolic) and maximal (peak systolic) diameters. With systolic expansion of lumen diameter, obligatory thinning of IMT will occur through conservation of mass (although some degree of longitudinal stretch will occur). Systematic timing of measurements is particularly important in serial study and/or intervention trials wherein the magnitude of change in measurements may approximate that seen in cyclic variation in IMT and lumen diameter.

IMT increases with age and, on average, is larger in men than women. In addition, modest racial differences in IMT have been reported. African Americans have higher CCA IMT values than Caucasians or non-Hispanic whites who, in turn, have slightly higher wall thicknesses than Hispanics. Ideally, age-, sex-, and race-adjusted thresholds derived from large population-based studies should be used to detect IMT.

The extent to which carotid IMT is a manifestation of early or diffuse atherosclerosis, as opposed to smooth-muscle hypertrophy and/or hyperplasia induced by pressure overload and/or age-related sclerosis, is uncertain. IMT has often been considered a manifestation of atherosclerosis, in view of the relations of IMT to cardiovascular disease (CVD) risk factors and to prevalent and incident CVD. Furthermore, in measurement protocols that allow incorporation of plaque thickness into the IMT measurement, IMT is, by definition, a measure of atherosclerosis. However, in protocols in which CCA IMT is measured with separate categorization of plaque, a clear dissociation between increased IMT and discrete carotid atherosclerosis has been demonstrated in relatively young patients with systemic lupus erythematosus, suggesting that IMT may not always represent a surrogate measure of atherosclerosis.

Internal diameter of the vessel lumen (usually the CCA) can be measured at a single point in time from B-mode images, or throughout the cardiac cycle from M-mode tracings. Determination of minimum and maximum lumen diameters is important for assessment of vascular mechanics. In addition, measurement of lumen diameter and IMT permits calculation of vascular cross-sectional area, a surrogate measure of vascular mass comparable with left ventricular mass. Such calculations may be particularly informative in intervention studies, particularly those using blood pressure-lowering agents.

Non-obstructive plaque, which may be defined as the presence of focal thickening at least 50% greater than that of the surrounding vessel wall, is usually readily identifiable, with the best view of its encroachment into the lumen detected from the transverse plane. The most common location of plaque is within the carotid bifurcation, when flow becomes less laminar, followed by the ICA; plaque is much less common in the CCA because of its usually laminar flow profile. Because Doppler velocity does not usually increase until significant (50%) luminal obstruction develops, nonobstructive plaque cannot be reliably quantified using Doppler techniques. Because of its complex 3-dimensional nature, the size of a single plaque or overall plaque burden is difficult to quantify; thus, the categoric presence of plaque is more reproducible than measurement of its thickness. Plaque diameter (ie, maximum incursion into the vessel lumen) may be measured. Some embodiments incorporate plaque diameter into IMT and do not make a distinction between IMT and plaque. In other embodiments, the number of discrete plaques, or the number of segments of the extracranial carotid arteries containing plaque, may be quantified.

In general, plaques may be characterized as homogeneous (ie, of uniform echogenicity) or as heterogeneous. Highly echogenic portions of heterogeneous plaques may correspond to areas of calcification, whereas echolucent areas may represent either lipid or hemorrhagic content. The presence of significant calcification is indicated by shadowing, or a signal void beyond the highly echogenic calcium. Quantitative tissue characterization using integrated backscatter analysis may be used to distinguish between predominantly fatty versus fibrous plaque. In another embodiments, detecting inflammation is used to characterize plaque content and activity is contrast-enhanced ultrasound. Activated leukocytes attached to the inflamed vessel wall may bind the shells of lipid microbubbles, which are detectable by ultrasound. Contrast enhancement of the carotid artery lumen-wall interface may improve the ease and accuracy of performing IMT measurements.

In certain embodiments, methods of the invention are used to measure the vascular endothelial function in the brachial artery. A patient is positioned supine with the arm in a comfortable position for imaging the brachial artery. A sphygmomanometric cuff is placed either above the antecubital fossa or on the forearm. Brachial artery impulse is palpated superomedial to the antecubital fossa. To obtain the brachial artery images, the transducer may be moved superomedially slowly from the antecubital fossa in a horizontal position with color flow Doppler turned on until the brachial artery and vein are seen in the transverse plane. The transducer is then rotated 90 degrees to perform imaging in the horizontal plane. Alternatively, the transducer may be placed in a vertical position along the lateral border of the biceps muscle, applying a firm constant pressure, and moved slowly medially until it slips in the medial groove of the biceps muscle, at which point a horizontal segment of brachial artery is seen at the bottom of the ultrasound screen. Minimal adjustment of the transducer allows the artery to be moved up toward the center of the image. The position of the transducer may be marked on the skin for future reference in the protocol. A segment with clear anterior and posterior intimal-lumen interfaces is selected for continuous 2-dimensional gray-scale imaging. Once the optimal artery image is obtained, the probe can be fixed in place using a stereotactic probe holder. The 2-dimensional image may be optimized using the depth function on the ultrasound system; alternatively, the zoom function may be used to magnify a selected segment of the brachial artery. Imaging depth and gain settings should be kept constant throughout the study. During image acquisition, anatomic landmarks such as veins and fascial planes are noted to help image the same segment of the artery throughout the study.

A pulsed wave Doppler recording is obtained from the midartery. Thereafter, arterial occlusion is created by cuff inflation to suprasystolic pressure. Typically, the cuff is inflated to at least 50 mm Hg above systolic pressure to occlude arterial inflow for 3 to 5 minutes. This causes ischemia and consequent dilation of downstream resistance vessels by autoregulatory mechanisms. Subsequent cuff deflation induces a brief high-flow state through the brachial artery (reactive hyperemia) to accommodate the dilated resistance vessels. The resulting increase in shear stress causes the brachial artery to dilate. The longitudinal image of the artery should be recorded continuously during cuff occlusion and for up to 2 minutes after cuff deflation. A midartery pulsed wave Doppler signal is obtained on immediate cuff release and no later than 15 seconds after cuff deflation to assess hyperemic velocity. The maximal increase in diameter occurs approximately 60 to 90 seconds after release of the occlusion cuff (FIG. 20).

The sphygmomanometer cuff may be placed above or below the antecubital fossa. When the cuff is placed on the upper part of the arm, reactive hyperemia typically elicits a greater percent change in diameter compared with that produced by the placement of the cuff on the forearm. This may be caused by a greater flow stimulus resulting from recruitment of more resistance vessels, or possibly by direct effects of ischemia on the brachial artery. Compared with forearm cuff occlusion, upper arm occlusion is technically more challenging for accurate data acquisition because of collapse of the brachial artery, nonvisualization of color Doppler flow, and the shifts in artery and soft tissue that occur on cuff release. Upper arm occlusion may also be less comfortable for the patient. Regular blood pressure cuffs may make imaging difficult because of minimal space for imaging, especially when the arm is short or obese. A more narrow blood pressure cuff overcomes these problems and also allows instantaneous cuff deflation, facilitating use by a single operator. The change in brachial artery diameter after cuff release increases as the duration of cuff inflation increases from 30 seconds to 5 minutes. The change in diameter is similar after 5 and 10 minutes of occlusion. The percent change in artery diameter decreases as the baseline vessel diameter increases.

Accurate analysis of brachial artery reactivity is highly dependent on the quality of ultrasound images obtained. Analysis may be performed by several methods. Software programs allow digitization of selected analog frames. Alternatively, a continuous digital recording may be obtained, or digital loops may be acquired at select time points. The boundaries for diameter measurements in the selected images (the lumen-intima or the mediaadventitia interfaces) are measured manually with electronic calipers or, preferably, automatically using edge-detection software. Brachial artery diameter should be measured at the same time in the cardiac cycle using electrocardiographic gating during image acquisition (FIG. 2). The onset of the R wave is used to identify end diastole, and the peak of the T wave reproducibly identifies end systole. For any given absolute change in the postflow stimulus diameter, a larger baseline diameter yields a smaller percent change. Thus, it is advisable to measure and report baseline diameter, absolute change, and percent change in diameter.

In certain embodiments, methods of the invention additionally involve internally obtaining characteristics of the body structure. Any method known in the art may be used to obtain the internal characteristics. In certain embodiments, the internal characteristics are obtained by imaging inside the body structure. Any intravascular imaging technique may be used. Exemplary imaging devices include optical coherence tomography (OCT), spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like. Alternatively or additionally, the internal characteristics may be obtained by fractional flow reserve.

In certain embodiments, the imaging device is an OCT device. OCT systems and methods are generally described in Castella et al. (U.S. Pat. No. 8,108,030), Milner et al. (U.S. Patent Application Publication No. 2011/0152771), Condit et al. (U.S. Patent Application Publication No. 2010/0220334), Castella et al. (U.S. Patent Application Publication No. 2009/0043191), Milner et al. (U.S. Patent Application Publication No. 2008/0291463), and Kemp, (U.S. Patent Application Publication No. 2008/0180683), the content of each of which is incorporated by reference in its entirety. Additional description of OCT systems and methods is described in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention include a light source. The light source may be any light source generally used with OCT. Exemplary light sources include a narrow line width tunable laser source or a superluminescent diode source. Examples of narrow line width tunable laser sources include, but are not limited to, lasers having a Bragg diffraction grating or a deformable membrane, lasers having a spectral dispersion component (e.g., a prism), or Fabry-Pérot based tuning laser.

OCT systems of the invention also include an interferometer. The interferometer may be any interferometer generally used with OCT. Typically, the interferometer will have a differential beam path for the light or a common beam path for the light. In either case, the interferometer is operably coupled to the light source. In a differential beam path layout, light from a broad band light source or tunable laser source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in Feldman et al. (U.S. Pat. No. 7,783,337) and Tearney et al. (U.S. Pat. Nos. 6,134,003 and 6,421,164), the content of each of which is incorporated by reference herein in its entirety.

The differential beam path optical layout of the interferometer includes a sample arm and a reference arm. The sample arm is configured to accommodate and couple to a catheter. The differential beam path optical layout also includes optical circulators to. The circulators facilitate transmission of the emitted light in a particular direction. Circulators and their use in OCT systems are further described for example in B. Bouma et al. (Optics Letters, 24:531-533, 1999), the entire disclosure of which is incorporated herein by reference. In the interferometer, there is a circulator where the emitted light is split to the sample arm and the reference arm. The system also includes a circulator that directs light to the sample and receives reflected light from the sample and directs it toward a detector. The system also includes a circulator that directs light to the reference surface and received reflected light from the reference surface and directs it toward the detector. There is also a circulator at the point at which reflected light from the sample and reflected light from the reference are recombined and directed to the detector.

In a common beam path system, rather than splitting a portion of the light to a reference arm, all of the produced light travels through a single optical fiber. Within the single fiber is a reflecting surface. A portion of the light is reflected off that surface prior to reaching a target tissue (reference) and a remaining portion of the light passes through the reflecting surface and reaches the target tissue. The reflected light from the tissue recombines with the signal from the reference forming interference fringes allowing precise depth-resolved imaging of the target tissue on a micron scale. Common beam path interferometers are further described for example in Vakhtin, et al. (Applied Optics, 42(34):6953-6958, 2003), Wang et al. (U.S. Pat. No. 7,999,938), Tearney et al. (U.S. Pat. No. 7,995,210), and Galle et al. (U.S. Pat. No. 7,787,127), the content of each of which is incorporated by reference herein in its entirety.

The common beam path optical layout of the interferometer includes a single array of optical fibers that are connected to a circulator. The array of optical fibers are configured to accommodate and couple to a catheter. The circulator directs light transmitted from the light source through the array of optical fibers of the common beam path optical layout to a sample and reference, and receives the reflected light from the sample and reference and directs it to the detector.

OCT systems of the invention include a detector. The detector includes photodetection electronics. The detector can support both balanced and non-balanced detection. OCT detectors are described for example in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention may conduct any form of OCT known in the art. One manner for conducting OCT may be Swept-Source OCT (“SS-OCT”). SS-OCT time-encodes the wavenumber (or optical frequency) by rapidly tuning a narrowband light source over a broad optical bandwidth. The high speed tunable laser sources for SS-OCT exhibit a nonlinear or non-uniform wavenumber vs. time [k(t)] characteristic. As such, SS-OCT interferograms sampled uniformly in time [S(t), e.g., using an internal digitizer clock] must be remapped to S(k) before Fourier transforming into the path length (z) domain used to generate the OCT image. An SS-OCT system and methods for its use are described in Kemp et al., (U.S. Patent Application Publication No. 2012/0013914). The content of which is incorporated by reference herein in its entirety.

In other embodiments, the imaging device is an IVUS device. There are two types of IVUS catheters commonly in use, mechanical/rotational IVUS catheters and solid state catheters. A solid state catheter (or phased array) has no rotating parts, but instead includes an array of transducer elements (for example 64 elements). In a rotational IVUS catheter, a single transducer having a piezoelectric crystal is rapidly rotated (e.g., at approximately 1800 revolutions per minute) while the transducer is intermittently excited with an electrical pulse. The excitation pulse causes the transducer to vibrate, sending out a series of transmit pulses. The transmit pulses are sent at a frequency that allows time for receipt of echo signals. The sequence of transmit pulses interspersed with receipt signals provides the ultrasound data required to reconstruct a complete cross-sectional image of a vessel.

The general design and construction of IVUS catheters is shown, for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et at., U.S. Pat. No. 4,917,097, Eberle et al., U.S. Pat. No. 5,135,486, Corl U.S. Pat. App. No. 2010/0234736, Davies et al., U.S. Pat. No. 8,317,713, Stephens et al., U.S. Pat. No. 6,780,157, Nix et al., U.S. Pat. No. 7,037,269, and other references well known in the art relating to intraluminal ultrasound devices and modalities. The catheter will typically have proximal and distal regions, and will include an imaging tip located in the distal region. Such catheters have an ability to obtain echographic images of the area surrounding the imaging tip when located in a region of interest inside the body of a patient. The catheter, and its associated electronic circuitry, will also be capable of defining the position of the catheter axis with respect to each echographic data set obtained in the region of interest.

Besides intravascular ultrasound, other types of ultrasound catheters can be made using the teachings provided herein. By way of example and not limitation, other suitable types of catheters include non-intravascular intraluminal ultrasound catheters, intracardiac echo catheters, laparoscopic, and interstitial catheters. In addition, the probe may be used in any suitable anatomy, including, but not limited to, coronary, carotid, neuro, peripheral, or venous.

In such embodiments, methods of the invention then involve combining external imaging data and internal data to produce an image of the body structure. Methods for combining external and internal image data are described, for example in Huennekens et al. (U.S. patent application number 2013/0030295), the content of which is incorporated by reference herein its entirety. IVUS imaging is exemplified in the below discussion, but methods of the invention are not limited to combinations with only IVUS imaging, and the principles discussed below can be used with any other internal imaging apparatus, such as OCT.

Creating, in a coordinated manner, graphical images of a body including vascular features from a combination of image data sources, in accordance with the present invention, includes initially creating an external ultrasound image of a vessel segment. The external ultrasound image is, for example, either a two or three dimensional image representation. Next, a vessel image data set is acquired that is distinct from the external ultrasound image data. The vessel image data set includes information acquired at a series of positions along the vessel segment. An example of such vessel image data is a set of intravascular ultrasound frames corresponding to circumferential cross-section slices taken at various positions along the vessel segment. The external ultrasound image and the vessel image data set are correlated by comparing a characteristic rendered independently from both the external ultrasound image and the vessel image data at positions along the vessel segment.

In FIG. 1, a diseased artery 5 with a lumen 10 is shown. Blood flows through the artery 5 in a direction indicated by arrow 15 from proximal end 25 to distal end 30. A stenotic area 20 is seen in the artery 5. FIG. 2 shows a sectioned portion of the stenotic area 20 of the artery 5. An artery wall 35 consists of three layers, an intima 40, a media 45 and an adventitia 55. An external elastic lamina (EEL) 50 is the division between the media 45 and the adventitia 55. A stenosis 60 is located in the artery 5 and limits blood flow through the artery. A flap 65 is shown at a high stress area 70 of the artery 5. Proximal to the stenosis 60 is an area of vulnerability 75, including a necrotic core 80. A rupture commonly occurs in an area such as the area of vulnerability 75.

FIG. 3 illustratively depicts an imaging catheter 85 having a distal end 95 that is inserted into the stenotic area 20 of the artery 5. The imaging catheter 85 is inserted over a guidewire 90, which allows the imaging catheter 85 to be steered to the desired location in the artery 5. As depicted in FIG. 4, the imaging catheter 85 includes an imaging element 100 for imaging the diseased portions and normal portions of the artery 5. The imaging element 100 is, for example, a rotating ultrasound transducer, an array of ultrasound transducer elements such as phased array/cMUT, an optical coherence tomography element, infrared, near infrared, Raman spectroscopy, magnetic resonance (MRI), angioscopy or other type of imaging technology. Distal to the imaging element 100 is a tapered tip 105 which allows the imaging catheter 85 to easily track over the guidewire 90, especially in challenging tortuous, stenotic or occluded vessels. The imaging catheter 85 can be pulled back or inserted over a desired length of the vessel, obtaining imaging information along this desired length, and thereafter creating a volumetric model of the vessel wall, including the diseased and normal portions, from a set of circumferential cross-section images obtained from the imaging information. Some technologies, such as IVUS, allow for the imaging of flowing blood and thrombus.

Known automatic border detection algorithms executed by an IVUS image data processing system facilitate identifying a luminal boundary. Plaque components are identified from information derived from IVUS radiofrequency backscatter and are color coded. The various characterized and graphically depicted plaque components potentially consist, by way of example, fibrous, fibro-lipidic (fibro fatty), necrotic core, calcified (dense calcium), blood, fresh thrombus, and mature thrombus. The RF backscatter can also give information to identify and color code stent materials such as metallic or polymeric stents. The distribution of components in a cross-section or in the entire volume of the vessel analyzed is displayed by way of example through various graphics. In addition to the cross-sectional display images rendered, a longitudinal is also that depicts information obtained from portions of a set of circumferential cross-sectional slides.

The external ultrasound image is obtained as described above. In accordance with an aspect of an imaging system embodying the present invention, IVUS images are co-registered with the three-dimensional image depicted on the graphical display. Fiduciary points are selected when the imaging catheter is at one or more locations, and by combining this information with pullback speed information, a location vs. time (or circumferential cross-sectional image slice) path is determined for the imaging probe mounted upon the catheter. Co-registering cross-sectional IVUS with three-dimensional external ultrasound images allows for a three-dimensional volumetric map of either gray scale images or colorized tissue characterization (tissue composition) images.

Turning to FIG. 5, a set of steps are depicted for creating a volumetric map. The particular order of the steps differs in alternative embodiments. During step 162, the imaging catheter is pulled back either manually or automatically through a blood vessel segment, and a sequence of circumferential cross-sectional IVUS image frames is acquired/created. During step 163 an external ultrasound image is formed of the blood vessel segment. The image is, for example, a two-dimensional image or, alternatively a three-dimensional image created from two or more external ultrasound views. During step 164, at least one fiduciary point is designated on the external ultrasound image, either by the user, or automatically by the imaging system. During step 166, the external ultrasound image and the information obtained from the imaging catheter during the pullback are aligned/correlated using the fiduciary point locating information. Thereafter, during step 168 the cross-sectional IVUS images are displayed on a graphical display in association with a two- or three-dimensional graphical representation of the imaged vessel. The graphical representation of the imaged vessel is based at least in-part upon the external ultrasound information. By way of example, in an exemplary embodiment, the external ultrasound itself is displayed. In an alternative embodiment, information from an external ultrasound is only used to guide piece-wise reconstruction of the imaged vessel from the sequence of IVUS image slices by determining the linear displacement and orientation of adjacent sections of the reconstructed vessel using the angiographic image of the vessel.

Turning to FIG. 6, by combining or overlaying the three-dimensional map of imaging information over the three-dimensional image 160 of the vessel lumen, or over one or more two-dimensional views of the external ultrasound image, a reconstruction 165 that more realistically represents the actual vessel is obtained, which is correct in its portrayal of vessel tortuosity, plaque composition and associated location and distribution in three dimensions. For example, a necrotic core which is located in the vessel in the sector between 30° to 90°, also having a certain amount of longitudinal depth, will appear on the reconstruction 165 with the same geometry. An augmented overall vessel diameter, due to thickened plaque, will also appear this way in the reconstruction 165. The additional information from the non-external ultrasound imaging data makes displaying such vessel images possible. The steps of the procedure summarized in FIG. 6 facilitate co-registration of the IVUS information over a live two-dimensional external ultrasound, giving the operator the ability to view a projection of the volume of plaque over a two-dimensional image of the lumen. The co-registered displayed graphical image allows an operator to make a more informed diagnosis, and also allows the operator to proceed with therapeutic intervention with the additional information provided by the co-registered displayed image guiding the intervention.

In the case of live two-dimensional or three-dimensional co-registration, one or more fiduciary points are selected first, followed by alignment by the system, and then simultaneous pullback and angiography or fluoroscopy. Note that in both co-registration in playback mode and co-registration in “live” mode, the information used by the system includes both the specific pullback speed being used (for example 0.5 millimeters per second) and the time vector of the individual image frames (for example IVUS image frames). This information tells the system where exactly the imaging element is located longitudinally when the image frame is (or was) acquired, and allows for the creation of an accurate longitudinal map.

Automatic fiduciary points are used, for example, and are automatically selected by the system in any one of multiple potential methods. A radiopaque marker on the catheter, approximating the location of the imaging element, for example is identified, creating the fiduciary point. Alternatively, the catheter has an electrode, which is identified by three orthogonal pairs of external sensors whose relative locations are known. By measuring field strength of an electrical field generated by the probe, the location of the electrode is “triangulated”.

FIG. 6 graphically depicts a reconstruction produced using the techniques discussed above. Three necrotic cores 80a, 80b and 80c have been identified. First necrotic core 80a is located at twelve o'clock circumferentially in the vessel and is identified as being located in the stenosis 60, and deep beneath a thickened cap. The location of the necrotic core 80a beneath the thickened cap suggests that this necrotic core is more stable than the other two necrotic cores—core 80b which is very close to the surface, and 80c which is also close to the surface. As shown in this reconstruction, and in relation to the first necrotic core 80a, the second necrotic core 80b is located at nine o'clock and the third necrotic core 80c is circumferentially located at four o'clock. This circumferential information is employed, for example, to localize application of appropriate treatment. The graphically depicted information provided by the imaging catheter and reconstruction allows delivery of the therapeutic catheter to the precise treatment location, with the desired catheter orientation. Alternatively, the imaging catheter itself is a combination imaging and therapy catheter, and the treatment simultaneously coincides with the imaging. One possible treatment scenario involves placing a drug eluting stent at the portion of the depicted vessel near the stenosis 60 and treating the second and third necrotic cores 80b and 80c by a needle-based drug, cell (i.e. stem cell) or gene delivery catheter (U.S. Pat. No. 6,860,867 to Seward), or by removing the necrotic core material by a needle and vacuum catheter. If using a tissue removal technique, such as atherectomy, ultrasonic therapeutics, or a plaque modification technique such as photodynamic therapy, drug delivery, radiation, cryoplasty, radiofrequency heating, microwave heating or other types of heating, the knowledge of the location of the EEL 50 is important. This assures that the adventitia is not disturbed, and that vessel perforation does not occur. The reconstruction 165 is graphically displayed in a manner that clearly demonstrates the location of the EEL 50 from all viewing angles. It can be seen that the thickness 170 between the luminal boundary and the EEL 50 at the stenosis 60 is much larger than the thickness 175 between the luminal boundary and the EEL 50 proximal to the stenosis 60. The circumferential (azimuthal) and radial (depth) orientation of the plaque components has been discussed herein above, but the axial (longitudinal) orientation/positioning—the distances separating diseased sections along a vessel's length—is important also. First necrotic core 80a is further distal than second necrotic core 80b, and second necrotic core 80b is further distal than third necrotic core 80c. The axial arrangement (lengthwise positioning) of diseased sections is important when choosing a particular length of a stent to use, or where to place the distal-most or proximal-most portion of the stent. It is also important when determining the order or operation in the treatment sequence. In addition, very proximally located vulnerable plaques are generally of greater concern than distally located vulnerable plaques, because they supply blood to a larger volume of myocardium.

Arteries also have side branches which can be identified with imaging techniques such as standard IVUS imaging, or IVUS flow imaging (which identifies the dynamic element of blood). The side branches are potentially used as fiduciary points for axial, circumferential and even radial orientation of the IVUS information, with respect to an angiographic base image, which also contains side branch information.

Turning to FIGS. 10-16, an exemplary technique is illustrated for obtaining accurate axial and circumferential co-registration of IVUS information (or other image information obtained via a probe inserted within a body) with the three-dimensional image 160. Turning initially to FIG. 10 and FIG. 11, the illustrations are intended to represent the internal representation of information created/processed by the imaging/display system. However, in an illustrative embodiment, such information is presented as well as graphical displays rendered by the system, in the manner depicted in FIGS. 10 and 11 as a visual aid to users in a semi-automated environment. For example, a user can manually move the relative positioning of a sequence of IVUS frames with regard to linear displacement of a vessel as depicted in corresponding data values generated from an angiographic image.

Furthermore, as those skilled in the art will readily appreciate, the line graphs in FIGS. 10 and 11 corresponding to IVUS frames comprise a sequentially ordered set of discrete values corresponding to a sequence of “N” frames of interest. Similarly, values generated from external ultrasound image data are also taken at discrete points along a length of a vessel of interest. Thus, while depicted as continuous lines in the drawing figures, the values calculated from external ultrasound and IVUS information correspond to discrete points along the length of the vessel.

FIG. 10 includes a graph 320 depicting calculated/estimated lumen area as a function of IVUS image frame number for both external ultrasound and IVUS. The graph depicted in FIG. 12 shows the effect of inaccurate co-registration between two imaging methods and associated measured parameters (e.g., lumen cross-section size). A line graph 330 representing lumen area calculated from IVUS information and a line graph 325 representing lumen area calculated from external ultrasound information are shown in an exemplary case wherein the measurements are misaligned along a portion of a vessel.

FIG. 10 corresponds to a graphically displayed composite image depicted in FIG. 12 that includes a graphical representation of a three-dimensional external ultrasound image 335 and a graphical representation of corresponding IVUS information 340 where the two graphical representations are shifted by a distance (“D”) in a composite displayed image. The misalignment is especially evident because minimum luminal circumferential cross-section regions (i.e., the portion of the vessel having the smallest cross-section) in the images graphically rendered from each of the two data sets do not line up. The minimal lumen area calculated from the IVUS information at point 345 in FIG. 10 corresponds to the IVUS minimal lumen position 360 in FIG. 12. The lumen area calculated from the external ultrasound information at point 350 in FIG. 10 corresponds to the external ultrasound minimal lumen position 355 in FIG. 12. Note that in the illustrative example, thickness of the vessel wall is depicted as substantially uniform on IVUS. Thus, an IVUS image frame where the minimal lumen area occurs is also where the minimum vessel diameter exists. This image feature differs from restricted flow due to a blockage within a diseased artery such as the one depicted in FIG. 2.

A lumen border 380 is also shown in FIG. 12. In order to achieve axial alignment between the graphical representation of the three-dimensional external ultrasound image 335 and the graphical representation of corresponding IVUS information 340, an axial translation algorithm is obtained based upon a “best-fit” approach that minimizes the sum of the squared differences between luminal areas calculated using the angiographic and the IVUS image data.

The best axial fit for establishing co-registration between angiogram and IVUS data is obtained where the following function is a minimum.

$${\sum }_{n = 1}^{N} {( {A}_{Lumen} - {A}_{Ex Ult} )}^{2}$$

with A_{Lumen}=IVUS lumen area for frames n=1, N and A_{Ex Ult} external ultraosund area for “frames” n=1, N (sections 1-N along the length of an angiographic image of a blood vessel). By modifying how particular portions of the external ultrasound image are selected, the best fit algorithm can perform both “skewing” (shifting all slices a same distance) and “warping” (modifying distances between adjacent samples).

Using the axial alignment of frames where the summation function is a minimum, a desired best fit is obtained. FIGS. 11 and 13 depict a result achieved by realignment of line graphs and corresponding graphical representations generated from the external ultrasound and IVUS data, depicted in a pre-aligned state in FIGS. 10 and 12, based upon application of a “best fit” operation on frames of IVUS image data and segments of a corresponding external ultrasound image,

FIG. 14 illustratively depicts a graphical representation of a three-dimensional lumen border 365 rendered from a sequence of IVUS image slices after axially aligning a three-dimensional external ultrasound data-based image with a graphical image generated from IVUS information for a particular image slice. The displayed graphical representation of a three dimensional image corresponds to the lumen border 380 shown in FIG. 13. The lumen border 380 is shown projected over a three-dimensional center line 385 obtained from the external ultrasound information. FIG. 14 also depicts a first external ultrasound image plane 370 and a second external ultrasound image plane 375 that are used to construct the three dimensional center line 385 and three-dimensional external ultrasound image 335. Such three-dimensional reconstruction is accomplished in any one of a variety of currently known methods. In order to optimize the circumferential orientation of each IVUS frame, an IVUS frame 400 depicting a luminal border is projected against the first external ultrasound plane 370, where it is compared to a first two-dimensional angiographic projection 390. In addition, or alternatively, the IVUS frame 400 is projected against the second external ultrasound image plane 375, where it is compared to the second two-dimensional external ultrasound projection 395 for fit. Such comparisons are carried out in any of a variety of ways including: human observation as well as automated methods for comparing lumen cross section images (e.g., maximizing overlap between IVUS and external ultrasound-based cross-sections of a vessel's lumen).

Positioning an IVUS frame on a proper segment of a graphical representation of a three-dimensional external ultrasound image also involves ensuring proper circumferential (rotational) alignment of IVUS slices and corresponding sections of an external ultrasound image. Turning to FIG. 15, after determining a best axial alignment between an IVUS image frame, such as frame 400, and a corresponding section of a three-dimensional external ultrasound image, the IVUS frame 400 is then rotated in the model by an angular displacement 405 (for example 1°), and the fit against the external ultrasound projections is recalculated. As mentioned above, either human or automated comparisons are potentially used to determine the angular displacement. After this has been done over a range of angular orientations, the best fit angular rotation is determined.

FIG. 16 depicts a graph 410 of best angle fit and frame number. During the pullback of the IVUS catheter, there may be some slight rotation of the catheter, in relation to the centerline of the blood vessel, and so, calculating the best angular fit for one IVUS frame does not necessarily calculate the best fit for all frames. The best angular fit is done for several or all frames in order to create the graph 410 including actual line 412 and fit line 414. The actual line 412 comprises a set of raw angular rotation values when comparing IVUS and external ultrasound circumferential cross-section images. The fit line 414 is rendered by applying a limit on the amount of angular rotation differences between adjacent frame slices (taking into consideration the physical constraints of the catheter upon which the IVUS imaging probe is mounted). By way of example, when generating the fit line 414, the amount of twisting between frames is constrained by fitting a spline or a cubic polynomial to the plot on the actual line 412 in graph 410.

FIGS. 7, 8 and 9 illustratively depict three different graphic displays for graphically representing information relating to plaque size and composition. A vessel lumen trace 260 is, for example, either a three-dimensional rendering of the vessel lumen (for example derived from two two-dimensional angiography images) or a two-dimensional projection of the three dimensional rendering. Alternatively, vessel lumen trace 260 is represented by a live external ultrasound image. In all of the aforementioned alternative external ultrasound imaging modes, it is possible to overlay images of the atherosclerotic plaque, however, it is difficult to appreciate the thickness, contours and composition of the plaque at all points extending circumferentially around the vessel by simply looking at a single projection.

FIG. 7 is a graphical image representation that embodies a technique that utilizes information calculated from IVUS imaging (or other imaging) and places a maximum thickness line 265 and a minimum thickness line 270 above and below the trace. Though not specific of where, circumferentially, the thickest portion of plaque occurs, the maximum thickness line 265 shows the exact maximum thickness of the plaque at each longitudinal position along the artery. In other words, a curving, continuous central axis parameter 275 follows the centerline of the artery and represents the axial location of the plaque, while a perpendicular axis parameter 280 represents the maximum thickness of the plaque by its distance from the edge of the vessel lumen trace 260. In a similar manner, the minimum thickness line 270 represents the minimum thickness of the plaque in the negative direction. It can be appreciated immediately while viewing the image/graphic combination depicted in FIG. 7 that the plaque is eccentric at various sections, even though there is no information present in this image/graphic combination to identify the exact circumferential angle where the maximum plaque thickness occurs. By viewing this image/graphic combination, the operator can immediately focus on the areas where the plaque is more eccentric, and the operator can also get a measurement of the minimum and maximum plaque thickness.

FIG. 8 illustratively depicts a graphical technique similar to that of FIG. 7, but with more specific information, namely the volume of plaque composition over a chosen length of vessel. A bar graph 285 is placed along-side the vessel lumen trace 260, and represents the volume of the different plaque components over a length of vessel. The user picks the proximal and distal point on the vessel which define a region of interest (for example a possible area of vulnerability), and the data obtained in this area is displayed with the bar graph 285. The bar graph 285 in this case represents four plaque components, fibrous 290, fibro-fatty 295, necrotic core 300, and dense calcium 305. The thickness (height in the radial direction) of each individual bar is proportional to the volume of that plaque component measured in a visually designated/indicated length of vessel. Each bar is color coded with a characteristic color to allow easier visual identification. For example, fibrous-dark green, fibrofatty-light green, necrotic core-red, dense calcium-white.

FIG. 9 illustratively depicts a graphical technique that is very similar to the one described in FIG. 7; however, instead of describing maximum and minimum plaque thickness at each axial location, the actual plaque thickness at each of the two sides is graphed. When the vessel lumen trace 260 is displayed in a two-dimensional mode, the upper thickness line 310 and the lower thickness line 315 graph the thickness of the plaque at points 180° from each other (for example at twelve o'clock and six o'clock), depending on the orientation chosen for the vessel lumen trace 260.

The invention described herein is not limited to intravascular applications or even intraluminal applications. Tissue characterization is also possible in cancer diagnostics, and it is conceivable that a probe that images for cancer can also be used in conjunction with a three-dimensional map to create a similar reconstruction as that described above. This can be used to guide biopsy or removal techniques. Such cancers include, but are not limited to: prostate, ovarian, lung, colon and breast. In the intravascular applications, both arterial and venous imaging is conceived. Arteries of interest include, by way of example: coronaries, carotids, superficial femoral, common femoral, iliac, renal, cerebral and other peripheral and non-peripheral arteries.

The intravascular ultrasound methods described can also be expected to be applicable for other ultrasound applications, such as intracardiac echocardiography (ICE) or transesophageal echocardiography (TEE). Therapeutic techniques that are guided by these techniques include, but are not limited to, patent foramen ovale closure, atrial septal defect closure, ventricular septal defect closure, left atrial appendage occlusion, cardiac biopsy, valvuloplasty, percutaneous valve placement, trans-septal puncture, atrial fibrillation ablation (of pulmonary veins or left atrium, for example) and TIPS (transjugular intrahepatic portosystemic shunt for pulmonary hypertension).

All of the techniques described here can also be used in conjunction with external imaging technologies such as MRI, CT, X-ray/angiography and ultrasound. Three dimensional reconstructions, for example from CT or MRI, can be co-registered with the imaging information in the same way as angiography.

It is also conceivable to include three-dimensional fluid mechanics analysis in the reconstruction so that points of high stress are identified.

A system of the invention may be implemented in a number of formats. An embodiment of a system 300 of the invention is shown in FIG. 19. The core of the system 300 is a computer 360 or other computational arrangement (see FIG. 20) comprising a processor 365 and memory 367. The memory has instructions which when executed cause the processor to receive imaging data of vasculature of a subject collected with an external image collector. The internal image data of vasculature will typically originate from a data collector 320, which is in electronic and/or mechanical communication with an internal imaging device 325. The memory additionally has instructions which when executed cause the processor to receive an image of the subject including the radiopaque label. The image of the subject will typically be an external ultrasound. The image of the subject will typically originate in an external imaging device 340, e.g., external ultrasound device. Having collected the images, the processor then processes the image, and outputs an image of the subject showing the location of the data collector 320, as well as an image of the vasculature of a subject. The images are typically output to a display 380 to be viewed by a physician or technician. In some embodiments a displayed image will simultaneously include both the internal ultrasound images of the vasculature and external ultrasound images of the vasculature, as described above.

In advanced embodiments, system 300 may comprise an imaging engine 370 which has advanced image processing features, such as image tagging, that allow the system 300 to more efficiently process and display combined internal and external vasculature map images. The imaging engine 370 may automatically highlight or otherwise denote areas of interest in the vasculature. The imaging engine 370 may also produce 3D renderings of the vasculature map images. In some embodiments, the imaging engine 370 may additionally include data acquisition functionalities (DAQ) 375, which allow the imaging engine 370 to receive the imaging data directly from the internal imaging device 325 to be processed into images for display.

Other advanced embodiments use the I/O functionalities 362 of computer 360 to control the intravascular imaging 320 or the external imaging modality 340. In these embodiments, computer 360 may cause the data collector of the internal imaging device 325 to travel to a specific location, e.g., if the internal imaging device 325 is a pull-back type. While not shown here, it is also possible that computer 360 may control a manipulator, e.g., a robotic manipulator, connected to internal imaging device 325 to improve the placement of the internal imaging device 325.

A system 400 of the invention may also be implemented across a number of independent platforms which communicate via a network 409, as shown in FIG. 20. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

As shown in FIG. 20, the internal imaging data collector 320 and the external imaging modality 340 are key for obtaining the data, however the actual implementation of the steps for data collection, co-registration of the data, storage and data output, can be performed by multiple processors working in communication via the network 409, for example a local area network, a wireless network, or the internet. The components of system 400 may also be physically separated. For example, terminal 467 and display 380 may not be geographically located with the internal imaging data collector 320 and the external imaging modality 340.

As shown in FIG. 20, imaging engine 859 communicates with host workstation 433 as well as optionally server (not shown) over network 409. In some embodiments, an operator uses host workstation 433, computer 360, or terminal 467 to control system 400 or to receive external imaging data and internal imaging data. An image may be displayed using an I/O 362, 437, or 471, which may include a monitor. Any I/O may include a monitor, keyboard, mouse or touch screen to communicate with any of processor 365,441, or 475, for example, to cause data to be stored in any tangible, nontransitory memory 367, 445, or 479. Server generally includes an interface module to communicate over network 409 or write data to data file. Input from a user is received by a processor in an electronic device such as, for example, host workstation 433, terminal 467, or computer 360. In certain embodiments, host workstation 433 and imaging engine 855 are included in a bedside console unit to operate system 400.

In some embodiments, the system may render three dimensional imaging of the vasculature or the intravascular images. An electronic apparatus within the system (e.g., PC, dedicated hardware, or firmware) such as the host workstation 433 stores the three dimensional image in a tangible, non-transitory memory and renders an image of the 3D tissues on the display 380. In some embodiments, the 3D images will be coded, as previously-discussed, for faster viewing. In certain embodiments, systems of the invention render a GUI with elements or controls to allow an operator to interact with three dimensional data set as a three dimensional view. In other embodiments an operator may select points from within one of the images or the three dimensional data set by choosing start and stop points while a dynamic progress view is displayed in display. In other embodiments, a user may cause an internal imaging device to be relocated to a new position in the body by interacting with the vasculature map image.

In some embodiments, a user interacts with a visual interface and puts in parameters or makes a selection. Input from a user (e.g., parameters or a selection) are received by a processor in an electronic device such as, for example, host workstation 433, terminal 467, or computer 360. The selection can be rendered into a visible display. In some embodiments, an operator uses host workstation 433, computer 360, or terminal 467 to control system 400 or to receive images. An image may be displayed using an I/O 362, 437, or 471, which may include a monitor. Any I/O may include a keyboard, mouse or touch screen to communicate with any of processor 365, 441, or 475, for example, to cause data to be stored in any tangible, nontransitory memory 367, 445, or 479. Server generally includes an interface module to effectuate communication over network or write data to data file. Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections). In certain embodiments, host workstation 433 and imaging engine 855 are included in a bedside console unit to operate system 400.

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer 360 having a graphical user interface or a web browser through which a user can interact with an implementation of the subject matter described herein), or any combination of such back-end, middleware, and front-end components. The components of the system can be interconnected through network 409 by any form or medium of digital data communication, e.g., a communication network. Examples of communication networks include cell networks (3G, 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include programming language known in the art, including, without limitation, C, C++, Perl, Java, ActiveX, HTML5, Visual Basic, or JavaScript.

A computer program does not necessarily correspond to a file. A program can be stored in a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over network 409 (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties so that optical read/write devices can then read the new and useful collocation of information (e.g., burning a CD-ROM). In some embodiments, writing a file includes using flash memory such as NAND flash memory and storing information in an array of memory cells include floating-gate transistors. Methods of writing a file are well-known in the art and, for example, can be invoked automatically by a program or by a save command from software or a write command from a programming language.

In certain embodiments, display 380 is rendered within a computer operating system environment, such as Windows, Mac OS, or Linux or within a display or GUI of a specialized system. Display 380 can include any standard controls associated with a display (e.g., within a windowing environment) including minimize and close buttons, scroll bars, menus, and window resizing controls. Elements of display 380 can be provided by an operating system, windows environment, application programming interface (API), web browser, program, or combination thereof (for example, in some embodiments a computer includes an operating system in which an independent program such as a web browser runs and the independent program supplies one or more of an API to render elements of a GUI). Display 380 can further include any controls or information related to viewing images (e.g., zoom, color controls, brightness/contrast) or handling files comprising three-dimensional image data (e.g., open, save, close, select, cut, delete, etc.). Further, display 380 can include controls (e.g., buttons, sliders, tabs, switches) related to operating a three dimensional image capture system (e.g., go, stop, pause, power up, power down).

In certain embodiments, display 380 includes controls related to three dimensional imaging systems that are operable with different imaging modalities. For example, display 380 may include start, stop, zoom, save, etc., buttons, and be rendered by a computer program that interoperates with internal imaging device and external imaging modalities, e.g., external ultrasound devices. Thus display 380 can display an image derived from a three-dimensional data set with or without regard to the imaging mode of the system.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of imaging a body structure, the method comprising:
providing an ultrasound device that is external to a patient's body; and
externally imaging a body structure within the patient using the external ultrasound device.

2. The method according to claim 1, further comprising constructing an image of the body structure.

3. The method according to claim 1, further comprising internally obtaining characteristics of the body structure.

4. The method according to claim 3, further comprising combining external imaging data and internal data to produce an image of the body structure.

5. The method according to claim 3, wherein the internal characteristics are obtained by imaging inside the body structure.

6. The method according to claim 5, wherein imaging is accomplished by a technique selected from the group consisting of intravascular ultrasound and optical coherence tomography.

7. The method according to claim 3, wherein the internal characteristics are obtained by fractional flow reserve.

8. The method according to claim 1, wherein the body structure is a vessel.

9. The method according to claim 8, wherein vessel is part of the patient's cardiovascular system.

10. The method according to claim 1, wherein the ultrasound emits a signal at about 20 megahertz or greater.

11. A method of imaging a vessel, the method comprising:
providing an ultrasound device that is external to a patient's body; and
externally imaging a vessel within the patient using the external ultrasound device.

12. The method according to claim 11, further comprising constructing an image of the vessel.

13. The method according to claim 11, further comprising internally obtaining characteristics of the vessel.

14. The method according to claim 13, further comprising combining external imaging data and internal data to produce an image of the vessel.

15. The method according to claim 13, wherein the internal characteristics are obtained by imaging inside the vessel.

16. The method according to claim 15, wherein imaging is accomplished by a technique selected from the group consisting of intravascular ultrasound and optical coherence tomography.

17. The method according to claim 13, wherein the internal characteristics are obtained by fractional flow reserve.

18. The method according to claim 11, wherein vessel is part of the patient's cardiovascular system.

19. The method according to claim 11, wherein the ultrasound emits a signal at about 20 megahertz or greater.
